SEQUENCE LISTING (7 sequences)

SEQ ID NO: 1 - DNA, 45 bp, Artificial Sequence
  synthetically generated oligonucleotide
gaaggcataagtttcttaactgggattattagctggggtgaagag

SEQ ID NO: 2 - DNA, 20 bp, Artificial Sequence
  synthetically generated oligonucleotide
tactgtcgtcgtcccctcaa

SEQ ID NO: 3 - DNA, 20 bp, Artificial Sequence
  synthetically generated oligonucleotide
ataactgccgtcactccaac

SEQ ID NO: 4 - DNA, 45 bp, Artificial Sequence
  synthetically generated oligonucleotide
aaggaagaaaagttttacaaaccgagcggtgtctgtgagactttc

SEQ ID NO: 5 - DNA, 45 bp, Artificial Sequence
  synthetically generated oligonucleotide
gaaagtctcacagacaccgctcggtttgtaaaacttttcttcctt

SEQ ID NO: 6 - DNA, 45 bp, Artificial Sequence; identical to SEQ ID NO: 5
  synthetically generated oligonucleotide

SEQ ID NO: 7 - DNA, 45 bp, Artificial Sequence; identical to SEQ ID NO: 4
  synthetically generated oligonucleotide

(19) United States
(12) Patent Application Publication
(10) Pub. No.: US 2012/0058180 A1
(43) Pub. Date: Mar. 8, 2012

(54) Liver-specific Nanocapsules and Methods of Using

(76) Inventors: Betsy T. Kren, Minneapolis, MN (US); Clifford J. Steer, St. Paul, MN (US); Gretchen M. Unger, Chaska, MN (US)

(21) Appl. No.: 13/221,552

(22) Filed: Aug. 30, 2011

Related U.S. Application Data
(63) Continuation of application No. 12/298,883, filed on Feb. 19, 2009, now abandoned.
(60) Provisional application No. 60/795,951, filed on Apr. 28, 2006.

(51) Int. Cl. A61K 9/51; A61K 38/48; A61K 38/37; A61K 31/713; A61P 1/16; A61P 3/00; A61P 7/00; A61P 31/14; A61P 37/00; A61P 11/00; A61P 35/00; A61K 31/711; A61P 7/04; B82Y 5/00

(52) U.S. Cl. 424/451; 514/44 R; 424/94.64; 514/14.1; 514/44 A; 977/773; 977/795; 977/915

(57) ABSTRACT

This disclosure describes liver-specific nanocapsules for specifically targeting liver cells. This disclosure also provides methods of using such liver-specific nanocapsules to deliver one or more cargo moieties to the liver cells.

### RELATED APPLICATIONS

This application is a continuation of U.S. patent application Ser. No. 12/298,883, filed on Feb. 19, 2009, which is a 35 USC §371 of PCT Application Serial No. PCT/US2007/067702, filed on Apr. 27, 2007, which claims priority to U.S. Provisional Patent Application No. 60/795,951, filed on Apr. 28, 2006, each entitled “Liver-Specific Nanocapsules and Methods of Using,” which are each incorporated herein in their entirety by reference.

### FEDERALLY SPONSORED RESEARCH OR DEVELOPMENT

The U.S. Government may have certain rights in this invention pursuant to Grant No. HL65578-01 awarded by National Institutes of Health (NIH).

### TECHNICAL FIELD

This invention relates to nanocapsules, and more particularly to liver cell-specific nanocapsules.

### BACKGROUND

Current non-viral delivery systems to the liver offer limited or no cell specificity. Moreover, non-specific delivery to tissues other than the liver is observed with the current delivery systems, as is activation of the host immune system. The present disclosure provides compositions that can be specifically targeted to one or more specific types of liver cells and methods of using such compositions. The compositions and the methods of the present disclosure do not activate the host immune system as do current delivery systems.

### SUMMARY

This disclosure describes a novel delivery system for targeting specific liver cells.

In one aspect, the invention provides a composition of nanocapsules comprising (or consisting essentially of) at least one liver-specific targeting moiety and at least one cargo moiety. Generally, the at least one targeting moiety is non-covalently associated with the nanocapsules and the at least one cargo moiety is encapsulated by the nanocapsules. Representative targeting moieties are an asialoorosomucoid (ASOR) polypeptide or a hyaluronan (HA) polypeptide.

In some embodiments, the at least one cargo moiety is a pharmaceutical agent. Representative pharmaceutical agents include, without limitation, a drug, a nucleic acid, a polypeptide, an anti-apoptotic agent, a chemoprotective agent, a chemopreventive agent, or an antiviral agent. For example, a nucleic acid can be a plasmid expressing a therapeutic polypeptide (e.g., Factor VII, a Factor VIII and a Factor IX polypeptide) or an oligonucleotide.

In another aspect, the invention provides methods of targeting nanocapsules to liver cells. Such methods generally include the steps of administering a composition of liver-specific nanocapsules to a subject, wherein the nanocapsules are targeted to and bind to liver cells.

In still another aspect, the invention provides methods of delivering a pharmaceutical agent to liver cells. Such methods generally include the steps of administering a composition of liver-specific nanocapsules to a subject. It is a feature of the invention that the nanocapsules are targeted to and bind to the liver cells, and that the binding of the nanocapsules to the liver cells results in the delivery of the pharmaceutical agent to the liver cells.

In certain embodiments, liver-specific nanocapsules can be administered intravenously or intraperitoneally. In such methods, the at least one targeting moiety can be ASOR polypeptides or HA polypeptides while the liver cells can be hepatocytes or liver sinusoidal endothelial cells (LSECs), respectively.

In yet another aspect, the invention provides methods of treating a subject having a disease of the liver. Such methods generally include the steps of administering a composition of liver-specific nanocapsules to a subject having a disease of the liver. It is a feature of the invention that the nanocapsules are targeted to and bind to liver cells and the binding of the nanocapsules to the liver cells results in the delivery of the pharmaceutical agent to the liver cells. Such methods thereby treat the subject having the disease.

Representative diseases of the liver include, without limitation, Crigler-najjar syndrome, hemophilia A or B, alpha-1-antitrypsin deficiency, Wilson's disease, familial hypercholesterolemia, maple syrup urine disease, ornithine transcarbamylase deficiency, phenylketonuria, lysosomal storage diseases, glycogen storage diseases, peroxisome diseases, familial amyloidosis, cytochrome p450 diseases, bile acid synthesis defects, non-alcoholic fatty liver disease, non-alcoholic steatohepatitis; hepatitis A, B, C, D or E; cirrhosis, hemachomatosis, autoimmune hepatitis; cystic fibrosis, or hepatocellular carcinoma (HCC). Similarly, representative pharmaceutical agents include, without limitation, an anti-viral agent, a recombinogenic oligonucleotide, a siRNA oligonucleotide, an antisense molecule, an episomal DNA plasmid, a protein, and a drug.

In another aspect, the invention provides for methods of mediating site-directed repair of a genomic mutation in liver cells of a subject. Such methods generally include the steps of administering a composition of liver-specific nanocapsules to the subject. It is a feature of the invention that the nanocapsules are targeted to and bind to the liver cells and the binding of the nanocapsules to the liver cells results in the delivery of the at least one cargo moiety to the liver cells. In one embodiment, the at least one cargo moiety is a single-stranded oligonucleotide, the delivery of which mediates site-directed repair of the genomic mutation in the liver cells of the subject.

Representative liver cells to which liver-specific nanocapsules can be targeted include, for example, hepatocytes and LSECs. Liver-specific nanocapsules can be administered, for example, intravenously or intraperitoneally. Genomic mutations can be, for example, point mutations. In embodiments of the invention, the liver cells, following administration of liver-specific nanocapsules, exhibit altered levels or activity of a polypeptide relative to the levels or activity of the polypeptide in the liver cells prior to administration. In further embodiments of the invention, the subject, following administration of liver-specific nanocapsules, exhibits improved phenotype compared to the subject prior to administration. It would be understood by those skilled in the art that the polypeptide is encoded by a nucleic acid sequence having homology to the single-stranded oligonucleotide cargo moiety. Representative polypeptides are clotting factor (e.g., Factor VII, Factor VIII, or Factor IX).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is micrographs showing expression analysis of nanoencapsulated LacZ transgene targeted to hepatocytes using ASOR (B and C), or targeted to LSECs with HA (A). The micrographs show characteristic blue color of cleaved X-gal substrate in the hepatocytes when [symbol] -galactosidase was expressed with hepatocyte specific SV40:alb promoter (B) or the constitutive SV40 promoter (C). No detectable expression of [symbol] -galactosidase in controls (D) or LSECs targeted using HA nanocapsules and hepatocyte promoter plasmid (A). No difference in expression or distribution of [symbol] -galactosidase was observed in the livers between male or female mice. Original magnification, 20×.

FIG. 2 shows agarose gels of PCR and RT-PCR analysis of DNA and RNA from livers of mice injected with LacZ ASOR or HA nanocapsules. (A) PCR of DNA and (B) RT-PCR of total RNA isolated from animals treated with HA or ASOR nanocapsules containing the prokaryotic [symbol] -galactosidase gene under control of the hepatocyte specific SV40:alb or constitutive SV40:ear promoters. Only mice treated with the ASOR-targeted nanocapsules expressed the prokaryotic LacZ mRNA in liver (345 bp band), but DNA encoding the transcript was present in all livers. Control mice exhibited no detectable signal for either the DNA or RNA. M, 100 base pair (282 bp) DNA ladder, lowest band shown 100 bp, increases in increments of 100 bp.

FIG. 3 shows agarose gels of PCR analysis of prokaryotic [symbol] -galactosidase coding sequence DNA present in tissues other than liver. Total DNA was isolated from testis, spleen, lung and kidney tissue from the mice that received the HA- and ASOR-targeted prokaryotic [symbol] -galactosidase expressing plasmids. PCR analysis was performed using 1 μg of DNA as template with the same primers and conditions as was used in FIG. 2A. No specific product was observed corresponding to the predicted size of base pairs (bp). The negative control (water without DNA) gave no product. M, 100 bp DNA ladder, lowest band shown 100 bp, increases in increments of 100 bp.

FIG. 4 shows RT-PCR transcript analysis of Gadd45 and Gadd153, genes involved in the global DNA damage response pathways. The RNA isolated from the excised neonatal liver was amplified by RT-PCR using primers specific for the mRNAs indicated above the gel. The animal groups are listed above their respective lanes and the size of the predicted fragments are indicated at left. M, 100 bp DNA ladder, lowest band shown 100 bp, increases in increments of 100 bp.

FIG. 5 shows the long-term histopathological analysis of ASOR-coated nanocapsules delivered in vivo to neonates. Representative micrographs of liver, kidney and spleen from neonates (n=3) injected with nanocapsules targeted to hepatocytes using ASOR (top), or untreated age matched controls (bottom). The micrographs show the histopathology of tissues three months post-treatment as 2 day neonates. No abnormal pathology was observed in any of the ASOR-treated livers, kidneys, spleen or lung relative to those isolated from the age matched untreated controls. The treatment group is indicated at left and the tissue above the panels. Original magnification, 40×; Bar 50 μm.

FIG. 6 is a schematic of the cis FVIII SB-Tn construct.

FIG. 7 is a graph showing the plasma aPTT levels in FVIII SB-Tn-treated mice. Results shown are the mean values±S.D. for each group of mice. *, p<0.001 compared to untreated mice.

FIG. 8 is a graph showing the blood loss following tail clipping. The mean values±S.D. are shown for the Factor VII treated hemophilia A mice (n=4) and wild-type controls (n=7). *, p<0.001 compared to untreated transgenic mice (˜3). Factor VIII alone and Factor VII+Factor VIII are the mean results (45% variance) from 2 mice that were treated in each group.

FIG. 9 shows a graph of the activated partial thromboplastin times (aPTT) in transgenic hemophilia A mice following treatment with a cytomegalovirus (CMV) enhancer:chicken beta-actin promoter hybrid (CAGGS) driven canine B-domain deleted Factor VIII (cFVIII) coding sequence (CDS) utilizing the rabbit beta-globin 3′UTR and poly adenylation (poly A) signal. The groups are indicated below. Wild-type, control age matched C57BL6 normal mice; Treated, Transgenic hemophilia A mice receiving 25 μg/20 g body weight s50 nm HA-targeted nanocapsules containing the cis CAGGS cFVIII Sleeping Beauty Tn (FIG. 1); Untreated, Transgenic hemophilia A mice. *, statistically significantly different using ANOVA and Bonferonni Multiple comparison test from untreated hemophilia A transgenic mice at p<0.001.

FIG. 10 shows luciferase expression in mouse pups after interperitoneal injection (ip) of ASOR-encapsulated CAGGS driven luciferase. One week following ip injection of 10 μg (10 mg/kg) of nanoencapsulated luciferse reporter plasmid targeted to hepatocytes using ASOR (top) or control tenfibgen nanocapsules (bottom), animals were imaged after the ip injection of the luciferin substrate. The results indicated that the ip-administered ASOR-coated nanocapsules were taken up and luciferase was expressed in the liver (top). In contrast, no signal was detected in the pups administered the control tenfibgen encapsulated luciferase plasmid (bottom).

FIG. 11 shows correction of a single point mutation in the canine Factor IX gene using ASOR-encapsulated 45-mer single-stranded oligonucleotides. The restriction fragment length polymorphism (RFLP) schematic is shown at the left, and the agarose gel analysis of the RFLP change resulting when the mutant A is corrected to the wild-type G in the genomic sequence is shown on the right. The arrow indicates the position of uncleaved amplicons prior to restriction endonuclease digestion using DdeI. Control, amplicons from untreated primary dog hepatocytes; treated, PCR amplicons of genomic DNA isolated from ASOR-45-mer treated hepatocyte cultures.

FIG. 12 shows the RFLP analysis of PCR amplicons spanning the OTC mutation target site. Genomic DNA isolated from livers of treated and both wild type and affected spf^{ash} untreated controls was amplified by PCR. The amplicons were subjected to DdeI digestion and analyzed by agarose gel electrophoresis. The treatment groups are indicated above the gels (top and middle) and the predicted size of the DdeI fragments in base pairs (bp) are indicated at left. A 100 bp ladder was used as a size standard, with the heavy band corresponding to 500 bp. Wt, wild type control; Mt, spf^{ash} affected control. The bottom panel shows the DNA sequence of one of the amplicons from a PEI-treated animal showing the mixture of A and G nucleotides at the targeted site, which is indicated by the *.

FIG. 13 shows PCR analysis of genomic regions sharing homology with the correcting 45-mer single-stranded oligonucleotides (SSOs). Relevant genomic sites were identified by BLASTn and amplified by PCR. The amplicons were subjected to direct sequence analysis and compared to the sequence from the reference assembly of the C57BL/6J mouse genome. The chromosome number and position are indicated above the text showing the SSOs in black or red font and the chromosomal region in blue font. The numbers in parentheses are the number of exact nucleotide matches, and the * indicate homologous nucleotides. The top panel shows the 45-mer wild type correcting OTC site (SEQ ID NO:6) with the complementary strand indicated in red for ease of identifying which of the complementary SSO sequences are aligned with the alternate chromosomal sites. The targeted mutation site at the OTC loci is indicated above the sequence panel by a 0; Ch7 (SEQ ID NO:4); Ch8 (SEQ ID NO:7); and Ch18 (SEQ ID NO:5).

Like reference symbols in the various drawings indicate like elements.

### DETAILED DESCRIPTION

This disclosure describes a nanocapsule vehicle for targeting specific liver cells. This disclosure also describes a novel therapeutic approach based upon the targeted delivery of a pharmaceutical agent (e.g., Factor VIII) to specific liver cells using such a nanocapsule vehicle. Such methods can be used to effectively correct hemophilia A, hepatitis, or other liver-associated disease due to, for example, a defective or absent gene product. The nanocapsule vehicles described herein are true capsules that carry the cargo within. The nanocapsules are less than 50 nm in size, even when carrying, for example, a relatively large cargo (e.g., a 15 Kb plasmid).

The prior art vehicles for liver-specific delivery are of limited utility because of recipient toxicity, low capacity for cargo delivery, and/or non-specific accumulation of the vehicle in either non-liver organs or reticuloendothelial elements. On the other hand, the nanocapsules described herein do not encounter many of the host-related complications that often result from introducing currently-available delivery vehicles (e.g., large nucleic acids). In addition, the neutral or net negative charge of the nanocapsules disclosed herein promotes long serum half-life and prevents the negative effects associated with, for example, positively-charged non-viral delivery vehicles such as accumulation of serum proteins via charge interactions, which ultimately increases the size of the capsule and thereby alters the tissue specificity and uptake.

### Nanocapsules

As used herein, nanocapsules refer to stabilized surfactant micelles having an average diameter of less than about 50 nanometers (i.e., “sub-50 nm nanocapsules”). Nanocapsules and methods of making nanocapsules are described, for example, in U.S. Pat. No. 6,632,671. The nanocapsules described herein can be targeted to the liver by coating the sub-50 nm nanocapsules with at least one liver-specific targeting moiety. “Coating” a nanocapsule with a targeting moiety refers to a non-covalent association between the nanocapsule and the targeting moiety.

A liver-specific targeting moiety can include, without limitation, an asialoorasomucoid (ASOR) polypeptide, a N-acetyl-galactosamine (NAG) sugar, an asialotrianntenary (A3) polypeptide or a hyaluronan (HA) polypeptide. Nanocapsules coated with ASOR, NAG, A3, arabinogalactan or another synthetic or naturally occurring galactose-presenting molecule specifically target hepatocytes via asialoglycoprotein receptors (ASGPr), while nanocapsules coated with HA, NAG or mannan specifically target liver sinusoidal endothelial cells (LSECs) via the hyaluronan, NAG or mannose receptors, respectively. Nanocapsules coated with targeting moieties such as ASOR and HA polypeptides result in highly efficient delivery of cargo to the respective liver cells while avoiding delivery of the cargo to liver Kupffer cells, which could result in toxic sequelae. Other targeting moieties such as NAG, A3, arabinogalactan or mannan result in less efficient delivery but can be used in situations where slower delivery and/or delivery to both hepatocytes and LSECs is warranted or desired, or in situations where delivery to non-liver tissues or organs, in addition to the liver, is not undesirable or harmful.

A cargo moiety can be any of a number of different compounds or molecules for imaging or monitoring purposes or for therapeutic purposes including, but not limited to, a pharmaceutical agent. A “pharmaceutical agent” as used herein refers to any compound or molecule that can be used to treat a disease or complication of the liver. A pharmaceutical agent can include, for example, a polypeptide, a nucleic acid molecule (e.g., a construct encoding a polypeptide, or an antisense RNA, RNAi, or siRNA nucleic acid molecule), an antiviral agent, a drug or small molecule (e.g., ursodeoxycholic acid and its amino acid conjugate, tauroursodeoxycholic acid and glycourodeoxycholic acid; s-adennosyl-L-methionine; 1-(isopropylamino) 3-(naphthalen-1-yloxy)propan-2-ol; hydroxyurea; or cortocosteriods), an anti-apoptotic agent, or a chemopreventive or chemoprotective agent. Experiments reported herein demonstrate that a cargo moiety delivered by a liver-specific nanocapsule of the invention exhibits a significantly longer half-life in the cell than cargo delivered using other vehicles. For example, expression of a nucleic acid construct delivered via a liver-specific nanocapsule as described herein is detectable for a number of weeks following administration, whereas expression of a nucleic acid using other delivery systems typically results in expression of the nucleic acid for only a few hours up to a few days.

The following is a brief description of the methods that can be used to make a liver-specific nanocapsule as disclosed herein. The following description is meant to be representative and is not meant to be limiting. Briefly, a negatively-charged moiety such as nucleic acid that is to be targeted and delivered to the liver can be complexed with a polycationic polymer to condense or reduce its size to about 50 nm or less. A number of different polycationic polymers (also known as “condensing” agents or proteins) can be used and are well-known in the art. See, for example, Rolland (1998, Crit. Rev. Therapeutic Drug Carr. Syst., 15:143-198). For example, enough complexing polycationic condensing protein can be used to neutralize at least about 75% (e.g., about 80%, 85%, 90%, 95%, 99% or 100%) of the negatively-charged cargo moiety, which, for nucleic acids, can be measured by ethidium dye exclusion (see, for example, Gershon (1993, Biochem., 32:7143-7151) as modified by Pouton (1998, J. Controlled Release, 53:289-99). Simply by way of example, 37.6 μg of 25 kD polyethyleneimine (PEI) can be used to condense 250 μg of a 7 kb DNA vector or 87.5 μg of 12,000 MW polyarginine can be used to condense 250 μg of an oligonucleotide. For cargo moieties lacking a negative charge, a condensing polycationic polymer may not be necessary.

The aqueous solution of the complexed or uncomplexed cargo moiety can be encapsulated by first dispersing the cargo moiety into a biocompatible, water-miscible solvent using a biocompatible, water-insoluble surfactant system suitable for preparation of an inverted or reverse micelle. Suitable surfactant systems are well-known in the formulation arts as amphillic materials that are essentially hydrophobic and characterized by a hydrophile-lipophile balance (HLB) of less than about 6, a critical micelle concentration (CMC) of less than about 200 μM, or a critical packing diameter greater than 1. Hydrophobic surfactants and hydrophobic, water-miscible solvents suitable for preparing reverse micelles are described in Pashley & Karaman (2004, In Applied Colloid and Surface Chemistry, John Wiley, pgs 60-85), Rosen (2004, In Surfactants and Interfacial Phenomena, John Wiley), The Handbook of Industrial Surfactants (1993, Ash, ed., Gower Pub), and Perry's Chemical Engineer's Handbook (1997, Perry & Green, 7^{th} Ed., McGraw-Hill Professional). In one embodiment, a hydrophobic surfactant can be 2,4,7,9-tetramethyl-5-decyn-4,7-diol (TM-diol) used in a concentration of up to 0.5% by weight of surfactant micelle volume, and a water-miscible solvent can be DMSO. The concentration of surfactant selected should be sufficient to prepare an optically clear nanoemulsion but not so much as to induce aggregation, since aggregation can lead to overly large nanocapsules.

The micelles carrying the cargo moieties (i.e., nanocapsules) can be coated with liver specific targeting moieties (e.g., ASOR or HA polypeptides) by mixing one or more targeting moieties with an aqueous dilution of the nanocapsules. Targeting moieties can be mixed with nanocapsules in a ratio (by weight) of about 1:100 to about 1:0.1 of nanocapsule to targeting moiety, depending upon the rate at which the nanocapsule is desired to dissolve or disassemble. In one embodiment, the coating weight ratio is 1:20 of nanocapsules to targeting moieties.

To stabilize the targeting moiety-adsorbed nanocapsule, the aqueous suspension of nanocapsules coated with one or more targeting moieties can be mixed into an aqueous solution of metal ions (i.e., a “stabilization solution”) capable of precipitating, crystallizing, or iontophoretic exchange with the coated nanocapsules. Representative and non-limiting examples of solutes that can be used to precipitate the coated nanocapsules include ionic species derived from elements listed in the periodic table. Ions may be included in the aqueous stabilization composition in a range from 0.1 ppb to 1 Molar (M). An adequate amount of ion should be included such that the coated nanocapsules are sufficiently contacted with ions but not so much that aggregation occurs, which can lead to overly large capsules. In one embodiment, a stabilization solution can include about 10 millimolar (mM) Ca²⁺ and about 200 mM Li⁺. If ultrapure reagents are used in the stabilization solution, addition of very small amounts (e.g., less than 1 mM) of ions such as Ba, Fe, Mg, Sr, Pb and Zn, normally found in sufficient quantities in more standard preparations of lithium and calcium salts, may be added to optimize stabilization of the coated nanocapsules. In one embodiment, a stabilization solution includes 9 mM Ca²⁺, 135 mM Li⁺, and 1-50 nM of Sr⁺³ and Mg⁺². Nanocapsules that have a final surface charge as close to neutral as possible or even slightly negative and/or that have the morphology of a compact or roughly spheroidal shape are indications of optimized stability. Additionally, any other components that are capable of increasing the stability of the nanocapsules can be included as part of the stabilization solution. Nanocapsules can be diluted into an aqueous solution of metal ions.

For a more consistent size of nanocapsules, the nanocapsules optionally can be atomized through a nozzle. Atomization should be sufficient to apply a shear force capable of breaking up flocculated aggregates without so much force as to induce hard aggregates. Those skilled in the art will understand that a particular nozzle diameter will lead to range of feed pressures suitable for atomizing the nanocapsules to a suitable and consistent size. In one embodiment, a nozzle diameter of less than about 250 microns with feed pressures of less than about 10 psi produces suitable nanocapsules. In some embodiments, the nanocapsules can be atomized into a stabilization solution.

The nanocapsules can be incubated in a stabilization solution for a few hours (e.g., 2.5, 5 or 8 hrs) up to several days (e.g., 2, 4, 6, 7, or 8 days) to vary the amount of time required for capsule dissolution or disassembly during end use. After precipitating, atomizing, and/or incubating the nanocapsules in a stabilization solution, the nanocapsules can be filtered, centrifuged and/or dried to obtain separate and discrete sub-50 nm nanocapsules. In one embodiment, nanocapsules are incubated for 2 days at about 4° C. The resultant nanocapsules can be frozen or dried and reconstituted for later use.

### Methods of Using Liver-Specific Nanocapsules

The liver is composed of many different cell types, only a few of which are desirable for therapeutic purposes. In particular, both the hepatocytes and the liver sinusoidal endothelia cells (LSECs) are desirable targets to modulate metabolic cellular activities or for gene therapy. On the other hand, there is very little therapeutic benefit to targeting and delivering a bioactive component to Kupffer or stellate cells in the liver. Thus, it is important to be able to target specific liver cells for effective liver-directed therapies, and the liver-specific nanocapsules described herein exhibit significantly improved targeting capability than other delivery vehicles (e.g., polylysine carriers) previously used in the art.

The liver-specific nanocapsules disclosed herein can be targeted specifically to liver cells, and can be used to deliver a cargo moiety (e.g., a pharmaceutical agent) to specific liver cells. A cargo moiety can be introduced into a nanocapsule during its production as described herein and as described in U.S. Pat. No. 6,632,671 (referred to as a “bioactive component” in the '671 patent).

A liver-specific nanocapsule can contain any cargo moiety that is useful for treating diseases or complications of the liver. A cargo moiety can be, for example, a pharmaceutical agent such as, without limitation, an antiviral agent for the treatment of hepatitis, a polypeptide or a nucleic acid to correct or replace a defective or missing gene product, or an antisense RNA, RNAi, or siRNA nucleic acid molecule for inhibiting the expression of a nucleic acid (e.g., encoding a deleterious polypeptide) in the respective liver cells. In addition, a pharmaceutical agent can include one or more drugs, one or more anti-apoptotic agents, or one or more chemopreventive or chemoprotective agents.

Liver-specific nanocapsules can be administered for targeting the liver by any number of different routes including, but not limited to, intravenous, intraperitoneal, oral, subcutaneous, intrathecal, intramuscular, inhalational, topical, transdermal, suppository (rectal), pessary (vaginal), intraurethral, intraportal, intrahepatic, intra-arterial, intra-ocular, transtympanic, intraumoral, intrathecal, transmucosal, buccal, or any combination thereof. The liver-specific nanocapsules described herein exhibit biocompatibility. As used herein, “biocompatible” refers to little or no toxicity (e.g., cytotoxicity), little to no undesired protein or nucleic acid modification or activation, or little to no induction of an undesired immune response. By way of example, mice administered a liver-specific nanocapsule as described herein did not display any toxicity, even in the absence of any tolerization.

Representative diseases or conditions of the liver that can be targeted using the liver-specific nanocapsules described herein include, but are not limited to, Crigler-najjar syndrome and other bilirubin diseases, hemophilia A and B, alpha-1-antitrypsin deficiency, Wilson's disease, familial hypercholesterolemia, maple syrup urine disease, ornithine transcarbamylase deficiency, phenylketonuria, lysosomal storage diseases, glycogen storage diseases, peroxisome diseases, familial amyloidosis, cytochrome p450 diseases, bile acid synthesis defects, and hepatocellular carcinoma (HCC).

For example, LSECs are the endogenous site of coagulation Factor VIII (FVIII) production and are involved in controlling the response to soluble circulating antigens. Thus, LSECs are an excellent target for directing replacement therapy of FVIII via either direct protein replacement and/or synthesis from an exogenously-introduced nucleic acid encoding the FVIII gene. In addition, hepatocytes are the site of ˜80% of the inborn metabolic errors in humans that are caused by defective or missing gene products, and hepatocytes are the cells that are infected with and that maintain the viral load of the hepatitis virus.

The liver-specific nanocapsules described herein also can be used for methods of mediating site-directed repair of a genomic mutation in liver cells of a subject. As described herein, a liver-specific nanocapsule carrying single-stranded oligonucleotides as cargo can be administering to a subject. Such liver-specific nanocapsules target and bind to liver cells and deliver the single-stranded oligonucleotide cargo, which mediates site-specific homologous recombination between a genomic mutation and the single-stranded oligonucleotide to repair the genomic mutation in the liver cells.

Genomic mutations that can be repaired using a liver-specific nanocapsule and the methods disclosed herein include, without limitation, point mutations (e.g., transitions (purine to purine or pyrimidine to pyrimidine) or transversions (purine to pyrimidine or vice versa)) and single- or multiple-nucleotide insertions or deletions. A mutation in a nucleic acid can result in one or more conservative or non-conservative amino acid substitutions in the encoded polypeptide, a shift in the reading frame of translation (“frame-shift”) resulting in an entirely different polypeptide encoded from that point on, a premature stop codon resulting in a truncated polypeptide (“truncation”), or a modification in a nucleic acid sequence may not change the encoded polypeptide at all (“silent” or “nonsense”). See, for example, Johnson & Overington, 1993, J. Mol. Biol., 233:716-38; Henikoff & Henikoff, 1992, Proc. Natl. Acad. Sci. USA, 89:10915-19; and U.S. Pat. No. 4,554,101 for disclosure on conservative and non-conservative amino acid substitutions.

Generally, following administration of the liver-specific nanocapsules described herein and the site-directed repair that such nanocapsules mediate, the liver cells exhibit altered levels or activity of a polypeptide relative to the levels or activity of the same polypeptide in the liver cells prior to administration. It is understood by those in the art that such a polypeptide is encoded by a nucleic acid sequence that has homology (or complementarity) to the single-stranded oligonucleotide cargo. In addition, following administration of the liver-specific nanocapsules described herein, the subject typically exhibits improved phenotype compared to the phenotype prior to administration of the nanocapsules.

For example, if a subject contains a genetic mutation in one or more clotting factors (e.g., Factor VII, Factor VIII, or Factor IX), a liver-specific nanocapsule as described herein containing a single-stranded oligonucleotide that has complementarity to a portion of the gene encoding the particular clotting factor can be administered to a subject. Following homologous recombination between the genomic DNA and the single-stranded oligonucleotide, the amount of the particular clotting factor is increased in the liver cells (as determined, for example, by immunoblotting (e.g., Western blot or ELISA)) and the subject typically demonstrates an improved phenotype (e.g., improved clotting). An number of genetic mutations, particularly those in hepatocytes, can be effectively repaired using the liver-specific nanacapsules described herein.

### Articles of Manufacture

The liver-specific nanocapsules described herein or some or all of the components required to make such liver-specific nanocapsules (e.g., targeting moieties and cargo moieties) can be provided in an article of manufacture. Articles of manufacture that include liver-specific nanocapsules or one or more components thereof can be provided, for example, in a dried (e.g., lyophilized), frozen or aqueous formulation.

An article of manufacture generally includes packaging material in addition to liver-specific nanocapsules or one or more components thereof. The packaging material can include a label or package insert that has instructions for treating an individual who has a disease of the liver. In addition, liver-specific nanocapsules can be formulated and/or packaged in dosage unit form for ease of administration and uniformity of dosage. Dosage unit form as used herein refers to physically discrete units suited as unitary dosages to be administered to a subject, with each unit containing a predetermined quantity of liver-specific nanocapsules and/or cargo moieties to produce the desired effect. A dosage unit form of liver-specific nanocapsules generally is dependent, for example, upon the desired concentration of cargo moieties in a subject and the route of administration.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### EXAMPLES

### Example 1

### Preparation of ASOR-Targeted Nanocapsules

This example describes how nanocapsules of the invention are generated. Nanocapsules for uptake, expression and therapeutic studies were prepared by the “dispersion atomization” method described in U.S. Pat. No. 6,632,671 with some modifications. Briefly, 250 μg of plasmid DNA was first complexed with 37.6 μg of 25 kDa polyethyleneimine (PEI; Sigma Chemical Co., St. Louis, Mo.), a branched cationic polymer, and dispersed into 150 μl of sterile water using a water-insoluble surfactant system (TM-diol, 7.5 μg in DMSO or SE-30 (Air Products)). The DNA used in these experiments was an 8.6 kb reporter plasmid contained a dsRed2 expression cassette under the control of a CMV promoter. Following emulsification with a water-miscible solvent (DMSO), the complexes were then inverted and diluted by the addition of 750 μl of PBS.

The resultant hydrophobic micelles were coated (non-covalently) by the addition of 6.3 μg of asialoorosomucoid (ASOR; prepared by the method of Stockert et al. (1980, Lab. Invest., 43:556-63); Formula A) then atomized into a LiCl salt receiving solution (135 mM Li⁺, 9 mM Ca²⁺, 500 nM Bi³⁺, 50 nM Sr²⁺, 50 nM Mg²⁺ (all ultrapure)). Following cold-room incubation (4° C.) with nominal rotation in 50 ml round-bottomed tubes, which stabilizes the coated micelles in the salt solution, the sub-50 nm nanocapsules were recovered by centrifugation at 20,000×g for 2 hrs and resuspended in PBS+10% lactitol (at a concentration of 0.5 μg/μl) for filter sterilization through a 0.2 μm filter. In all formulations described, a small amount (1% of coating weight) of Syrian hamster IgG was “spiked” into the ligand coat to enable immunodetection of nanocapsules uptake by anti-syrian hamster IgG antibodies.

Average capsule size was less than 50 nm as measured by tapping mode atomic force microscopy using elliptical diameters of a 1 ng/ml sample dried down on a mica sheet. A surface charge of −8.6±2.8 mev was measured on Zetasizer 4 dynamic light scattering device at a potential of 20 volts with a 2-second pause between measurements in 1 mM KCl at 2 μg/ml.

### Example 2

### Sub-50 nm Nanocapsule Formulas

The targeting polypeptides used in these experiments were the ASOR polypeptide, a n-acetyl-galactosamine (NAG) sugar molecule, or asialotrianntenary (A3) polypeptide, all of which are recognized by the aisaloglycoprotein receptors (ASGPr) on hepatocytes (a hepatocyte-specific nanocapsule), or a hyaluronan (HA) polypeptide, which is a ligand that specifically recognizes the hyaloronan receptors (HAr) on liver sinusoidal endothelial cells (LSECs) (a LSEC-specific nanocapsule).

Formula A: the sub-50 nm nanocapsules coated with ASOR were described in Example 1.

Formula B: sub-50 nm nanocapsules coated with N-acetyl galactosamine (NAG) were generated as described in Example 1 except that 6.5 mcg of NAG (obtained from Sigma) was added to 250 mcg of a 8.6 kb cis Sleeping Beauty transposon (SB-Tns) plasmid containing the DsRed2 gene driven by the CMV promoter (CMVSB10pT2DsRed2) and condensed with 37.5 mcg of 25 kD PEI. Average capsule size was less than 50 nm as measured by tapping mode atomic force microscopy using elliptical diameters of a 1 ng/ml sample dried down on a mica sheet. A surface charge of −9.5±5.9 mev was measured on Zetasizer 4 dynamic light scattering device.

Formula C: sub-50 nm nanocapsules coated with asialotrianntenary (A3; V-labs, Covington, Iowa) were generated as described in Example 1 except that 6.5 mcg of triantennary peptide was added to 250 mcg of a 8.6 kb cis Sleeping Beauty transposon (SB-Tns) plasmid containing the DsRed2 gene driven by the CMV promoter (CMVSB10pT2DsRed2) and condensed with 37.5 mcg of 25 kD PEI. Average capsule size was less than 50 nm as measured by tapping mode atomic force microscopy using elliptical diameters of a 1 ng/ml sample dried down on a mica sheet.

Formula D: sub-50 nm nanocapsules coated with hyaluronan (HA) were generated as described in Example 1 except that 6.5 mcg of HA (1 mM kD; obtained from Lifecore Biomedical, Chaska Minn.) was added to 250 mcg of a 8.6 kb cis Sleeping Beauty transposon (SB-Tns) plasmid containing the DsRed2 gene driven by the CMV promoter (CMVSB10pT2DsRed2) and condensed with 37.5 mcg of 25 kD PEI. When generating these nanocapsules, the Sr⁺³ in the stabilization solution was modified to 62.5 nM and the Mg²⁺ was modified to 25 nM. Average capsule size was less than 50 nm as measured by tapping mode atomic force microscopy using elliptical diameters of a 1 ng/ml sample dried down on a mica sheet, and a surface charge of −4.6±4.5 mev was measured on Zetasizer 4 dynamic light scattering device.

Formula E: sub-50 nm nanocapsules coated with HA were generated as described in Example 1 except that 3.2 mcg of HA (1 mM kD) was added to 250 mcg of a 5.2 kb plasmid containing a prokaryotic β-galactosidase LacZ transgene controlled by the hepatocyte-specific hybrid SV40 enhancer:albumin promoter (pdriveAlbSV40-LacZ) and condensed with 36.6 mcg of 25 kD PEI. When generating these nanocapsules, Bi⁺³ was removed in the stabilization solution, Sr⁺³ was modified to 60 nM and Mg²⁺ to 5 nM, and capsules were incubated for 24 hours before centrifugation. Average capsule size was less than 50 nm as measured by tapping mode atomic force microscopy using elliptical diameters of a 1 ng/ml sample dried down on a mica sheet.

Formula F: sub-50 nm nanocapsules coated with HA were generated as described in Example 1 except that 3.2 mcg of HA (1 mM kD) was added to 250 mcg of a 6.8 kb plasmid containing the LacZ gene under control of the constitutive SV40 enhancer and early (SV40:ear) promoter (pSV40:ear/LacZ) and condensed with 37.5 mcg of 25 kD PEI. When generating these nanocapsules, Bi⁺³ was removed from the stabilization solution, Sr⁺³ was modified to 26.3 nM and Mg²⁺ was modified to 7.5 nM, and capsules were incubated for 24 hours before centrifugation. Average capsule size was less than 50 nm as measured by tapping mode atomic force microscopy using elliptical diameters of a 1 ng/ml sample dried down on a mica sheet.

Formula G: sub-50 nm nanocapsules coated with ASOR were generated as described in Example 1 except that 6.3 mcg of ASOR was added to 250 mcg of a 5.2 kb plasmid containing the LacZ gene under control of the hepatocyte-specific hybrid SV40 enhancer:albumin (SV40Alb, 5.2 kb) promoter (pdriveAlbSV40-LacZ) and condensed with 36.6 mcg of 25 kD PEI. When generating these nanocapsules, Bi⁺³ was removed from the stabilization solution, Sr⁺³ was modified to 42 nM and Mg²⁺ was modified to 14 nM, and capsules were incubated for 24 hours before centrifugation. Average capsule size was less than 50 nm as measured by tapping mode atomic force microscopy using elliptical diameters of a 1 ng/ml sample dried down on a mica sheet.

Formula H: sub-50 nm nanocapsules coated with ASOR were generated as described in Example 1 except that 6.3 mcg of ASOR was added to 250 mcg of a 6.8 kb plasmid containing the LacZ gene under control of the constitutive SV40 enhancer and early (SV40:ear) promoter (SV40:ear/LacZ) and condensed with 36.6 mcg of 25 kD PEI. In generating these nanocapsules, Bi⁺³ was removed from the stabilization solution, Sr⁺³ was modified to 42 nM and Mg²⁺ was modified to 14 nM, and capsules were incubated for 24 hours before centrifugation. Average capsule size was less than 50 nm as measured by tapping mode atomic force microscopy using elliptical diameters of a 1 ng/ml sample dried down on a mica sheet.

Formula I: sub-50 nm nanocapsules coated with HA were generated as described in Example 1 except that 12.5 mcg of HA (1 mM kD) was added to 250 mcg of a 12 kb plasmid containing Factor VIII under control of the hybrid CMV enhancer:chicken β-actin (CAGGS) promoter (pT2/caggs/F8/IFSB10) and condensed with 38.7 mcg of 25 kD PEI. When generating these nanocapsules, Bi⁺³ was removed from the stabilization solution, Sr⁺³ was modified to 37.5 nM and Mg²⁺ was modified to 12.5 nM, and capsules were incubated for 48 hours before centrifugation. Average capsule size was less than 50 nm as measured by tapping mode atomic force microscopy using elliptical diameters of a 1 ng/ml sample dried down on a mica sheet.

Formula J: sub-50 nm nanocapsules coated with HA are prepared as described in Example 1 except that 12.5 mcg of HA (1 mM kD) are added to 500 mcg of a Factor8 protein (Calbiochem) with 37.5 ug of TM-diol without condensation. When generating these nanocapsules, Bi⁺³, Li, Sr and Mg are removed from the stabilization solution, and calcium ion concentration is modified to 26.8 mM. Capsules are incubated for 14.5 hours before centrifugation. Average capsule size is less than 50 nm as measured by tapping mode atomic force microscopy using elliptical diameters of a 1 ng/ml sample dried down on a mica sheet.

Formula K: sub-50 nm nanocapsules coated with ASOR were generated as described in Example 1 except that 6.3 mcg of ASOR was added to 250 mcg of a 5 kb plasmid containing a gene encoding Factor VII under control of the hepatocyte-specific hybrid SV40 enhancer:albumin promoter (IFHSB3/1pkt2/sv40albF7#11) and condensed with 36.6 mcg of 25 kD PEI. When generating these nanocapsules, Bi⁺³ was removed from the stabilization solution, Sr⁺³ was modified to 27.5 nM and Mg²⁺ was modified to 10 nM, and capsules were incubated for 48 hours before centrifugation. Average capsule size was less than 50 nm as measured by tapping mode atomic force microscopy using elliptical diameters of a 1 ng/ml sample dried down on a mica sheet.

Formula L: sub-50 nm nanocapsules coated with HA is manufactured as described in Example 1 except that 12.5 mcg of HA (1 mM kD) is added to 250 mcg of a 6.9 kb plasmid expressing Factor IX under control of the hepatocyte-specific SV40:alb promoter (sv40albIFHSB3pKT2 CMVEFIalphaF9#1b) and condensed with 37.5 mcg of 25 kD PEI. When these nanocapsules are generated, Bi+3 is removed from the stabilization solution, Sr+3 is modified to 12.5 nM and Mg2+ is modified to 2.5 nM, and capsules were incubated for 36 hours before centrifugation. Average capsule size is less than 50 nm as measured by tapping mode atomic force microscopy using elliptical diameters of a 1 ng/ml sample dried down on a mica sheet.

Formula M: sub-50 nm nanocapsules coated with HA were generated as described in Example 1 except that 6.3 mcg of HA (1 mM kD) was added to 250 mcg of a 7.8 kb plasmid encoding human α1AT under the direction of a hybrid CMV enhancer:elongation factor 1α (EF1α) promoter (pT2cisAT) and condensed with 37.5 mcg of 25 kD PEI. When generating these nanocapsules, Br³ was removed from the stabilization solution, Sr⁺³ was modified to 12.5 nM and Mg²⁺ was modified to 2.5 nM, and capsules were incubated for 48 hours before centrifugation. Average capsule size was less than 50 nm as measured by tapping mode atomic force microscopy using elliptical diameters of a 1 ng/ml sample dried down on a mica sheet.

Formula N: sub-50 nm nanocapsules coated with ASOR were generated as described in Example 1 except that 6.3 mcg of ASOR was added to 250 mcg of a 7.2 kb plasmid encoding the luciferase gene under control of the hybrid CMV enhancer:chicken β-actin (CAGGS) promoter (pT2Luc5a) and condensed with 37.5 mcg of 25 kD PEI. When generating these nanocapsules, Bi⁺³ was removed from the stabilization solution, Sr⁺³ was modified to 12.5 nM and Mg²⁺ was modified to 2.5 nM, and capsules were incubated for 36 hours before centrifugation. Average capsule size was less than 50 nm as measured by tapping mode atomic force microscopy using elliptical diameters of a 1 ng/ml sample dried down on a mica sheet.

Formula O: sub-50 nm nanocapsules coated with ASOR were generated as described in Example 1 except that 12.5 mcg of ASOR was added to 500 mcg of a 45-mer oligo (5′-GAA GGC ATA AGT TTC TTA ACT GGG ATT ATT AGC TGG GGT GAA GAG-3′ (SEQ ID NO:1)) targeted to repair the G to A missense mutation in the Factor IX gene in the Chapel Hill model of canine hemophilia B and condensed with 87.5 μg of 12,000 MW polyarginine (Sigma). When generating these nanocapsules, Bi⁺³, Sr and Mg were removed from the stabilization solution, and capsules were incubated for 14.5 hours before centrifugation. Average capsule size was less than 50 nm as measured by tapping mode atomic force microscopy using elliptical diameters of a 1 ng/ml sample dried down on a mica sheet.

### Example 3

### Tissue Specificity of the Nanocapsules of Formulas A, B, C and D

To examine tissue specificity in vivo, eight week (wk) old (˜20 g) C57/BL6 mice received 100 μg of the ASOR-coated nanocapsules of Formula A, the NAG-coated nanocapsules of Formula B, the A3-coated nanocapsules of Formula C, or the HA-coated nanocapsules of Formula D via tail-vein injection and were sacrificed at 1 wk post-injection. The liver, spleen, kidneys, lungs, heart and brain were excised and a portion of each organ was processed for histology while proteins were extracted from another portion of each organ.

Immunohistochemical identification of the LSECs in cryosections was done using anti-CD14 antibody (Ab), a marker specific for the discontinuous endothelial cells in the liver, and a Cy5-labeled secondary Ab. Additional cryosections for hepatocytes were processed by staining of the nuclei with SYTOX® green (Molecular Probes) and visualized by confocal microscopy. The confocal micrographs showed the Cy5-labeled LSECs with the DsRed2 fluorescence.

The merged confocal micrographs of the DsRed2 and Cy5 fluorescence demonstrated co-localization of DsRed2 expression and the LSEC-specific CD14 marker when HA was used as the targeting ligand (i.e., with LSEC-specific nanocapsules). In contrast, there was no detectable co-localization when ASOR was used for targeting DsRed2 to hepatocytes (i.e., with hepatocyte-specific nanocapsules). There was no detectable DsRed2 expression with either the hepatocyte-specific (ASOR-coated) or the LSEC-specific (HA-coated) nanocapsules in the other major organs examined. With NAG- and A3-coated nanocapsules, uptake and expression in the liver as well as in other organs such as the kidneys was observed.

The presence of the DsRed2 protein also was confirmed by Western blot analysis. Total liver protein extracts were separated on a 12% PAGE, transferred to nylon membranes and detected using enhanced chemiluminescence (ECL) with a rabbit polyclonal anti-DsRed2 Ab (BD Clontech). Only the liver extracts from mice treated with either the hepatocyte-specific nanocapsules or the LSEC-specific nanocapsules expressed the DsRed2 reporter protein, while control mice exhibited no detectable signal. The Western blot analysis of the other tissues confirmed that the HA and ASOR nanocapsules only targeted the DsRed2 to the liver, as none of the other tissue extracts had detectable DsRed2 protein. However, with NAG- and A3-coated nanocapsules, DsRed2 protein was detected in the kidneys and at a very low level in the spleen.

### Example 4

### Tissue Specificity of the Nanocapsules of Formula E, F, G, or H

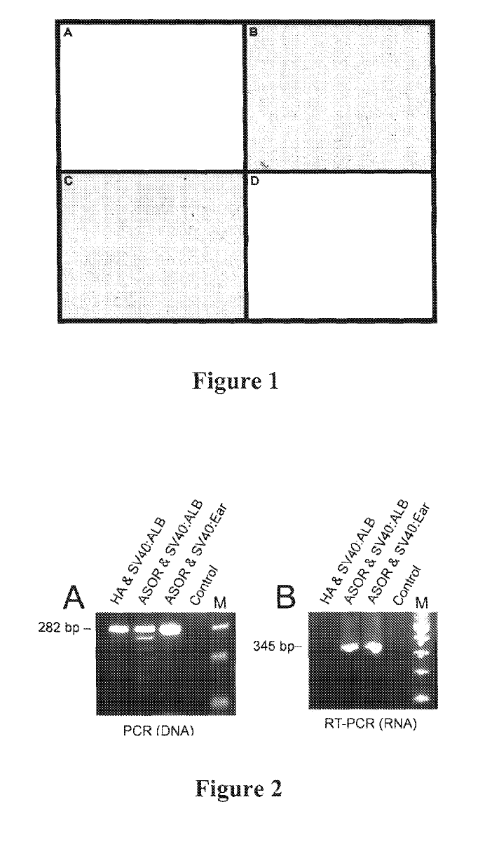

To further examine tissue specificity in vivo, eight week old—20 g C57/BL6 male and female mice were administered 100 μg of the ASOR-coated nanocapsules of Formula G or H or the HA-coated nanocapsules of Formula E or F via tail vein injection and sacrificed 1 week post-injection. The ASOR-targeted nanocapsules contained LacZ driven by either the SV40:alb or the SV40:ear promoter, while the HA-targeted nanocapsules contained LacZ under the control of the SV40:alb promoter. Liver, kidney, spleen, lung, heart and testes were removed and DNA and RNA was isolated from a portion of each organ. Another portion of the liver was used for cryosections of 10 μM, histochemically stained using X-Gal (5-bromo-4-chloro-3-indolyl-β-d-galactopyranoside) and visualized by light microscopy (FIG. 1).

DNA was isolated from livers and 0.25 μg was used as template for PCR amplification of β-galactosidase coding sequence using primers 5′-TAC TGT CGT CGT CCC CTC AA-3′ (SEQ ID NO:2) and 5′-ATA ACT GCC GTC ACT CCA AC-3′ (SEQ ID NO:3). HA-coated nanocapsules delivered 3-galactosidase coding sequence DNA to LSECs (FIG. 2A). No expression of β-galactosidase was detected in HA-targeted LSECs with the SV40:alb promoter (FIG. 2B) based on RT-PCR using 0.5 μg of DNase-treated total RNA isolated from the same livers as template. PCR and RT-PCR confirmed the presence of the β-galactosidase DNA and RNA transcript in the livers of all mice treated with the ASOR-coated nanocapsules that target hepatocytes (FIG. 2A, 2B).

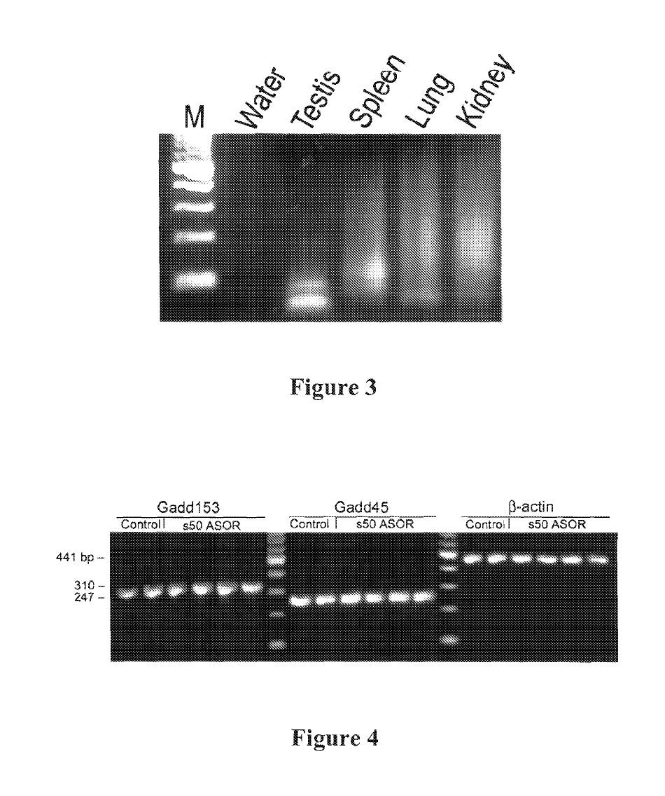

Total DNA also was isolated from lung, kidney, spleen and testes from the mice administered the ASOR- and HA-coated nanocapsules containing β-galactosidase-expressing plasmids, and 1 μg was used as template DNA for PCR using the same primers and conditions as FIG. 2A. No plasmid DNA was detected in any the non-liver organs examined (FIG. 3).

These experiments establish that cell type-specific targeting of the nanocapsules could be achieved when either ASOR or HA was used to coat the nanocapsules, and that the hybrid SV40:alb promoter exhibits hepatocyte-specific expression in liver. These experiments also demonstrate that dual delivery to both cell types can be accomplished with specificity.

### Example 5

### Safety Profile #1

The safety profile of the ASOR- and HA-coated nanocapsules was examined in an additional group of mice. The animals received 100 μg of the encapsulated DsRed2 SB-Tns targeted to the hepatocytes or LSECs using ASOR (n=2) or HA (n=7), respectively, by tail vein infection in a volume of 200 μl. The animals were sacrificed 72 hours post-injection, and blood was collected and analyzed. The results are shown in Tables 1 and 2, and indicate that the nanocapsules did not significantly alter the blood chemistry values relative to control mice (n=3).

TABLE 1
Standard blood chemistries

| Treatment | Alb g/dL | ALT U/L | AST U/L | UN mg/dL | TP g/dL |
|---|---|---|---|---|---|
| Controls | 2 ± 0.1 | 62 ± 8 | 358 ± 13 | 14 ± 3 | 4.6 ± 0.1 |
| Nanocapsule | 2 ± 0.2 | 57 ± 12 | 310 ± 99 | 13 ± 2 | 4.5 ± 0.3 |

Nanocapsule, sub-50 nm nanocapsules coated with HA or ASOR;
Alb, albumin;
UN, urea nitrogen;
TP, total protein.

TABLE 2
Hemogram

| Treatment | WBC | RBC | Hg | Hc | MCV | MCH | MCHC | RDW | PC |
|---|---|---|---|---|---|---|---|---|---|
| Control | 0.55 ± 0.1 | 8.4 ± 0.2 | 13.8 ± 0.1 | 40.7 ± 0.9 | 49 ± 0.7 | 16.5 ± 0.5 | 33.9 ± 1.1 | 17.0 ± 0.6 | 962 ± 111 |
| Nanocapsule | 0.66 ± 0.3 | 8.0 ± 0.3 | 13.4 ± 0.5 | 39.9 ± 1.3 | 50 ± 1.1 | 16.7 ± 0.3 | 33.5 ± 0.6 | 17.5 ± 1.1 | 1034 ± 67 |

Nanocapsule, s50 nm nanocapsules with HA or ASOR;
WBC, WBC count × 10⁹/L;
RBC, RBC count × 10¹²/L;
Hg, hemoglobin g/dL;
Hc, hematocrit %;
MCV, MCV fl;
MCH, MCH pg;
MCHC, g/dl;
RDW, RDW %;
PC, platelet count × 10⁹/L.

### Example 6

### Safety Profile #2

To examine the safety of ASOR-coated nanocapsules administered neonatally, pups ˜54 hrs in age received 10 mg/kg body weight of the nanocapsules of Formula G by temporal facial vein injection. No adverse effects of acute toxicity (e.g., death) were observed. Forty-eight hrs after injection of the capsules, 4 treated and 2 control pups were sacrificed and the livers removed for DNA damage analysis using an RT-PCR strategy developed by Smith et al. (2006, J. Gene Med., 8:175-85). Briefly, transcript levels of Gadd45 and Gadd153, key genes involved in the cellular response to global DNA damage, and β-actin mRNA as the normalization control were measured using 0.5 μg of liver total RNA as template. No increase in the levels of either Gadd transcript was observed in the treated group relative to controls (FIG. 4). This data indicates that ASOR-coated nanocapsules do not upregulate expression of genes involved in the DNA damage response.

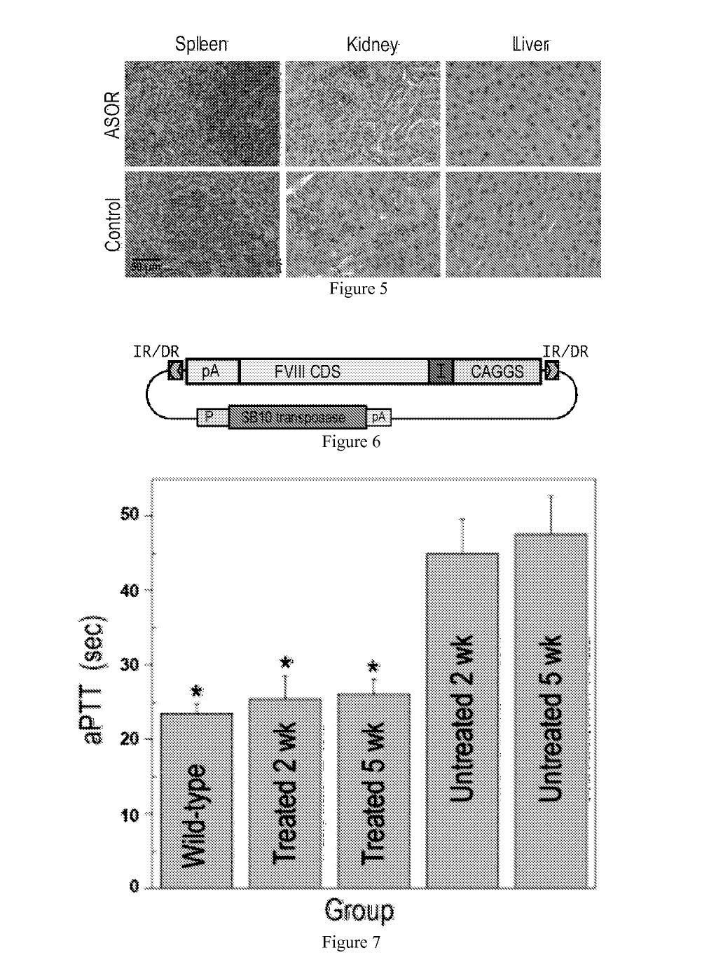

To investigate the long term safety of neonatally administered ASOR-coated nanocapsules, two day old C57/BL6 male and female mice were administered 10 mg/kg of prokaryotic β-galactosidase nanoencapsulated plasmid targeted to the hepatocytes using ASOR via temporal facial vein injection and sacrificed 3 months post-injection. The tissues were fixed, paraffin-embeded, and 4 μM sections were stained with hematoxylin and eosin for histopathological evaluation. The liver, kidney and spleen from three control and from ASOR-coated nanocapsule-treated animals were visualized by light microscopy. No abnormal pathology was observed in any of the livers, kidneys, or spleen from animals treated with the ASOR-coated nanocapsules relative to those isolated from the age-matched untreated controls (FIG. 5).

### Example 7

### In Vivo Targeting of Liver Cells

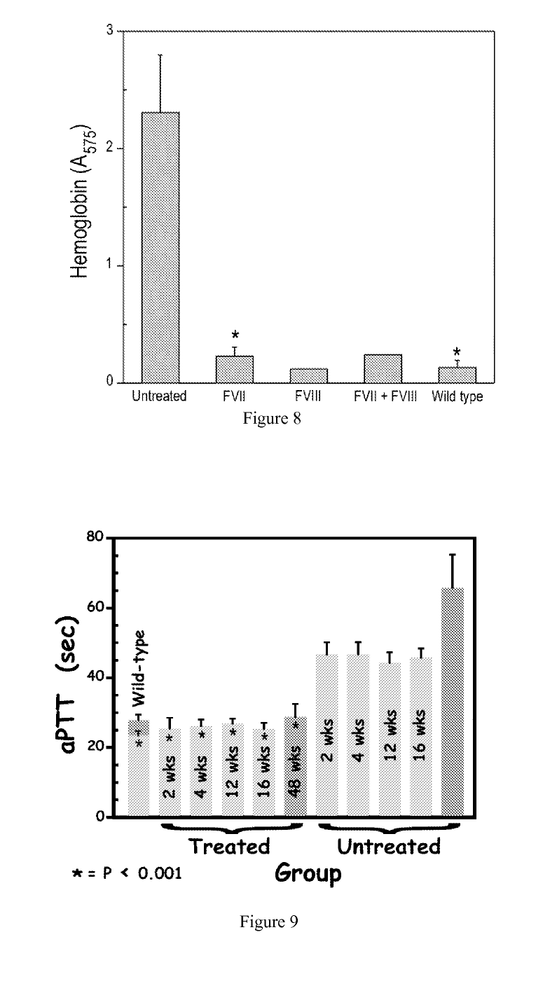

Transgenic hemophilia A mice were administered 25 μg of the LSEC-specific nanocapsules containing a Sleeping Beauty (SB) transposon (Tn) expressing B-domain deleted coagulation factor (F) VIII using a plasmid (pT2/caggs/F8/IFSB10) that co-delivers an expression cassette for the SB transposase required for genomic Tn insertion (cis FVIII SB-Tn; FIG. 6) by tail vein injection. The treated mice (n=6) and the wild type (wt) (n=6) and untreated (n=3) controls were bled 2 and 5 wks post-injection and their plasma-activated partial thromboplastin time (aPTT; Diagnostica Stago ST4 semi-automated hemostasis analyzer) (FIG. 7) and clotting profile (FIG. 8) was analyzed.

The treated mice had aPTTs of 25.5±3.1 sec at 2 wks and 26±1.9 sec at 5 wks, which were not significantly different from the wt aPTT of 23.5±1.3 sec. In contrast, untreated hemophilia A mice had aPTTs of 46.7±3.5 sec (p<0.001 from treated and wt mice). Therefore, the aPTTs in the treated animals were reduced to almost wt control levels by 2 wks, and this remained unchanged at 5 wks. In contrast, the untreated animals had prolonged aPTTs, which were significantly different (p<0.001) from wt or treated mice.

These experiments demonstrated that ASOR- and HA-coated nanocapsules delivered plasmids in vivo specifically to hepatocytes or LSECs, respectively. SB-Tns in LSEC-specific nanocapsules provided expression of a clinically relevant gene product, Factor VIII (FVIII), that improved the phenotype of hemophilia A transgenic mice. These results indicated that LSEC-targeted expression of FVIII can significantly improve the observed bleeding diathesis in the hemophilia A mice.

### Example 8

### Successful Systemic Treatment without Inhibitor Formation for Hemophilia

To test functional activity of the SB-Tns in vivo, cis SB-Tns (pT2/caggs/F8/IFSB10) expressing the B-domain deleted canine FVIII were encapsulated in HA nanocapsules targeted for delivery to LSECs (Formula I). Hemophilia A mice were administered 25 μg of the cis FVIII SB-Tns (n=6) in a 100 μl volume by tail vein injection. The animals have been followed using activated partial thromboplastin times (aPTT) for 11 months post-injection. The results (FIG. 9) demonstrate a significant decrease in aPTT in the FVIII-treated animals relative to the untreated transgenic mice (n=3) (p<0.001) at all time points. In fact, ANOVA analysis using Bonferroni multiple comparison tests indicated that the clotting time in the FVIII-treated animals did not differ significantly from the wild type controls (n=6) through 11 mos (dark gray). By 11 mos, however, all untreated controls were dead and the aPTT in the untreated controls from the same colony remained significantly different from the treated animals, which exhibited no significant difference in aPTT from the wild-type controls.

As an adjunct treatment, FVIII protein may be delivered to LSECs using HA-coated nanocapsules in which with the recombinant FVIII protein itself is encapsulated. These capsules are prepared as described in Formula J. Formula dosages are consistent with the current guidelines for recombinant FVII administration from the National Hemophilia Foundation Medical and Scientific Advisory Council (MASAC Document #175).

ASOR- or HA-coated nanocapsules (Formula K or L) containing Factor VII (FVII) or Factor IX (FIX) also were delivered to correct the bleeding diathesis observed in hemophilia A and/or hemophilia B. FIG. 8 shows the reduction of aPTT in hemophilia A mice receiving FVII via ASOR-coated nanocapsules.

### Example 9

### HA-Coated Nanocapsules Carrying α1-Antitrypsin Mediate Secretion of Active Hepatocyte Proteins by LSECs

Alpha1-antitrypsin (α1AT)-treated female mice (n=5) received 50 μg of the nanocapsules of Formula M via tail vein injection. The animals were bled, the blood was clotted and spun, and the serum levels of α1AT in the targeted LSECs were determined at 1 and 3 wks post-injection using the α1AT enzyme immunoassay (ALPCO Diagnostic). The levels of α1AT in the treated animals were 375±45 ng/ml and 390±65 ng/ml at 1 and 3 wks, respectively.

In summary, this data supports the cell type-specific targeting of the nanocapsule delivery system using the ASOR ligand for targeting hepatocytes and the HA ligand for targeting LSECs. Results from these experiments confirmed the hepatocyte-specific expression of the hybrid SV40:alb promoter even when the transgene was delivered to LSECs via HA targeting. The animal data in which an α1AT-expressing transgene was delivered indicates that the LSECs are a functional target cell type for expressing secreted proteins even if LSECs are not the native site of production for the protein. In addition, the data from the transgenic hemophilia A animals demonstrates that the phenotype associated with the disease in this animal model can be reversed by delivering a FVIII transgene using the ASOR- or HA-coated nanocapsule without inhibitory antibody formation.

### Example 10

### ASOR-Coated Nanocapsules Carrying a Single-Stranded Oligonucleotide Deliver Specifically to Liver Hepatocytes in Neonates and Mediate Gene Repair of Point Mutations

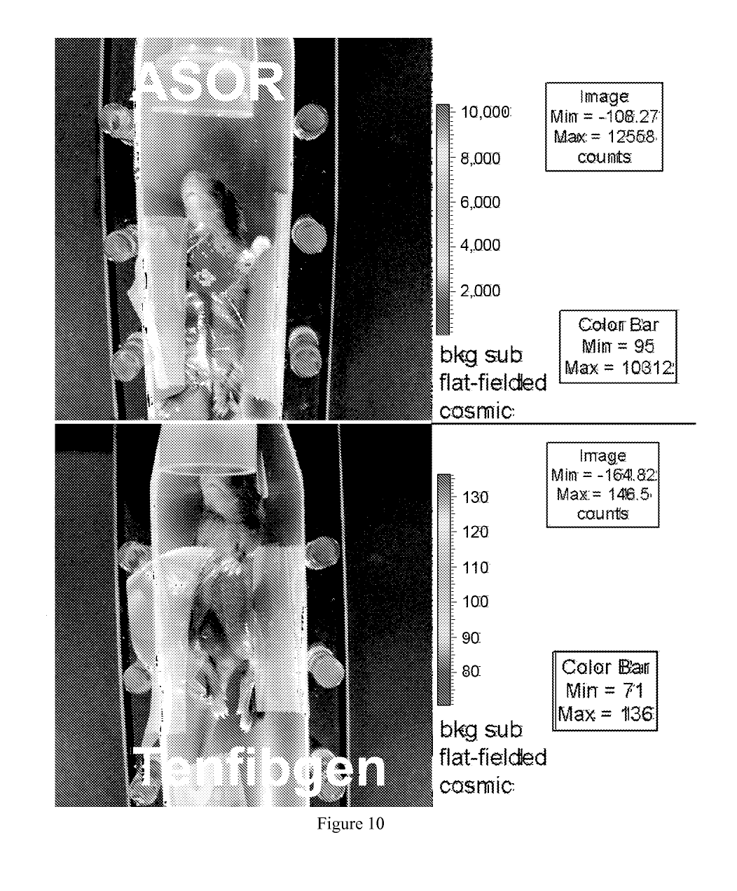

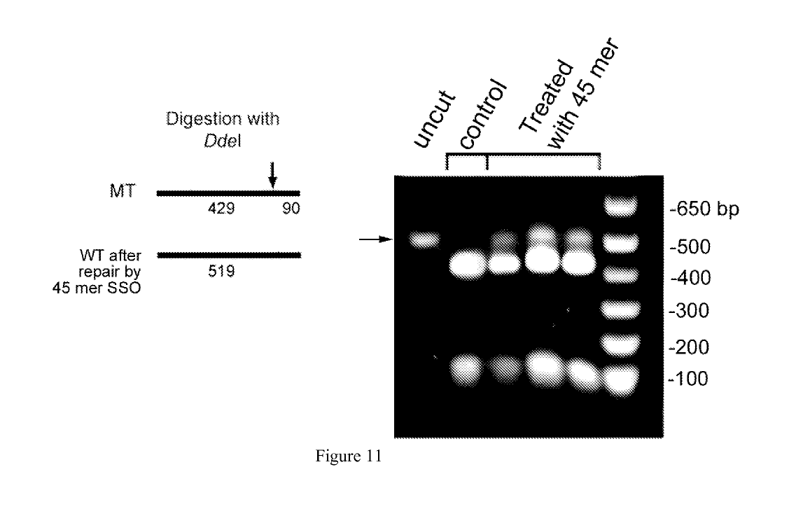

The following experiments determined that the ASOR-coated nanocapsules were taken up by hepatocytes when injected intraperitoneally (ip). Three day-old pups were injected ip with 10 μg of the nanocapsules of Formula N or 10 μg of the same plasmid nanoencapsulated using tenfibgen, a tumor specific targeting ligand. One week after injection, luciferase expression in the mouse pups was determined in vivo using a Zenogen imaging system following i.p. injection of the luciferin substrate. The results indicated that ip-administered ASOR-coated nanocapsules were taken up and luciferase and expressed in the liver (FIG. 10, left). In contrast, no luciferase signal was detected in pups administered the tenfibgen-coated nanocapsules carrying the luciferase plasmid (FIG. 10, right), even when imaging was extended to 5 mins.

To determine if a 45-mer single-stranded oligonucleotide could function in gene repair of a specific hepatocyte target diseases such as hemophilia B, primary hepatocytes from Chapel Hill canine hemophilia B animals were transfected with 5 μg of the nanocapsule of Formula 0 and harvested 8 days later. The DNA was isolated and PCR amplification of the gene spanning the targeted A to G change (mutant to wild-type gene sequence) was performed. This change also alters a restriction endonuclease cleavage site and thus introduces a restriction fragment length polymorphism (RFLP) difference between the wild-type (does not cut with DdeI) and mutant (unrepaired allele, cuts with DdeI). The data shown in FIG. 11 indicates that the 45-mer in the ASOR-coated nanocapsule promoted the desired A to T change in the Factor IX genomic sequence in the host cells.

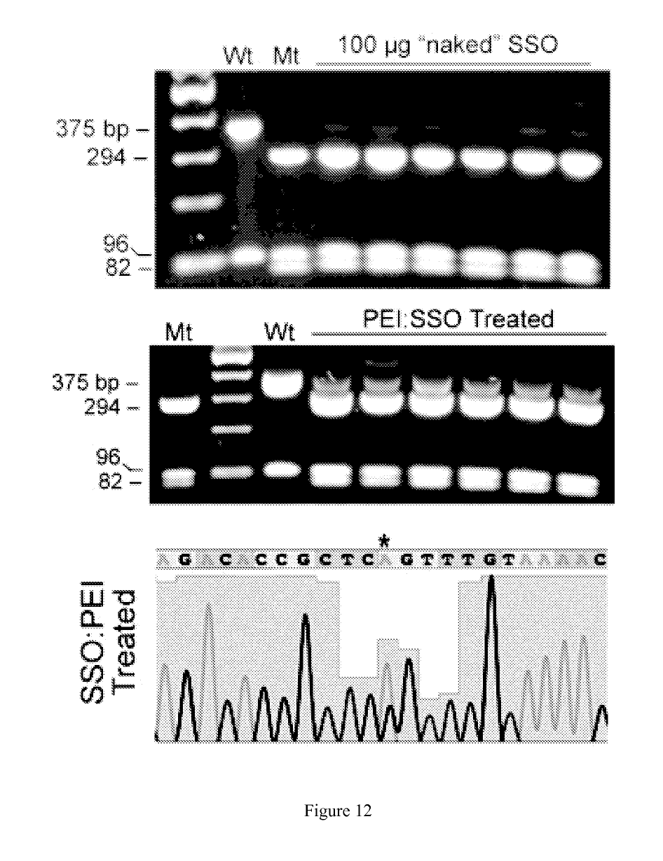

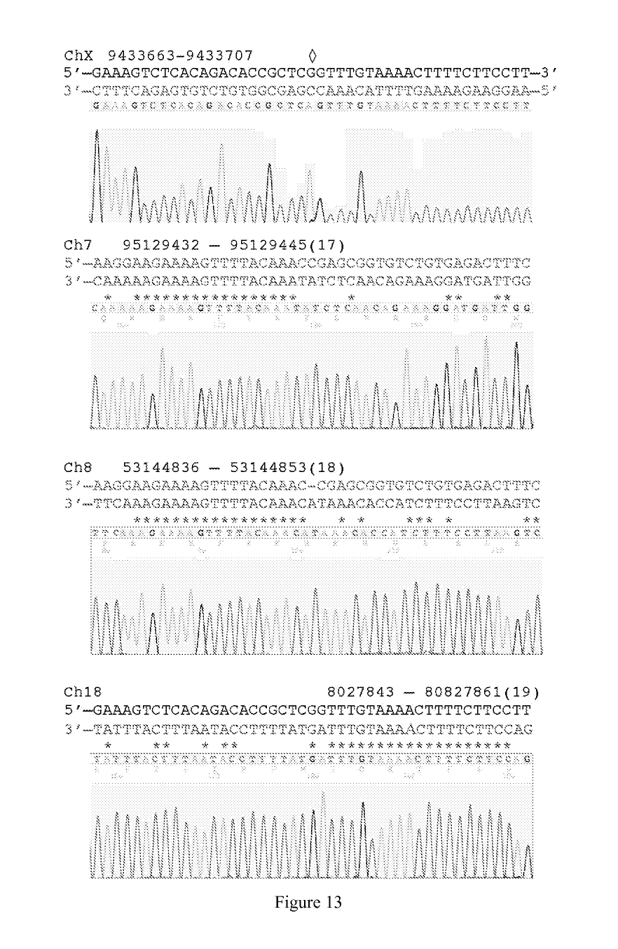

Ornithine transcarbamylase (OTC) deficiency results in high levels of ammonia in the blood and significant mental and developmental illnesses result if the affected individuals survive the first year of life. Treatment as soon as possible after birth or in utero to correct the genetic point mutation in the liver that results in the disease is desirable. These experiments demonstrate the ability of single-stranded oligonucleotides to target and correct the gene mutation in neonatal transgenic spf/ash mice, a model of this human disease. 45-mer oligonucleotides spanning the CGT to CAT missense mutation in the mouse gene were administered by temporal facial vein injection either without encapsulation (‘naked’, 100 μg) or using lactosylated PEI (20 μg) targeted to the ASGPr on hepatocytes. These 45-mers (5′-AAG GAA GAA AAG TTT TAC AAA CCG AGC GGT GTC TGT GAG ACT TTC-3′ (SEQ ID NO:4) or 5′-GAA AGT CTC ACA GAC ACC GCT CGG TTT GTA AAA CTT TTC TTC CTT-3′ (SEQ ID NO:5)) were either complementary to the transcribed or non-transcribed strand of the OTC sequence. Using these 45-mers, the desired single nucleotide change was mediated (FIG. 12), with increased body weight and enzyme activity in treated animals compared to untreated age-matched controls (Tables 3 and 4). No changes were observed at closely other related sequences in the genome (FIG. 13).

TABLE 3
Effect on Neonates of the Naked 45-mer to Correct the
spf/ash Mutation in OTC-Deficient Mice

| | Female Body | OTC activity (nmol |
|---|---|---|
| Group | Weight (g) | citrulline/μg protein/30 min) |
| Affected | 21.8 ± 4.6 (n = 6) | 0.78 ± 0.02 (n = 6) |
| spf/ash | | |
| Treated spf/ash | 27.1 ± 3.8 (n = 5) | 1.36 ± 0.21 (n = 7) |
| Unaffected | 28.8 ± 2.4 (n = 6) | 37.03 ± 2.93 (n = 6) |

TABLE 4
Effect on Neonates of Administration of
Lactosylated PEI Complexed with a 45-mer
for Repair of the OTC Loci in spf/ash Mice

| | Body | OTC activity (nmol citrulline/ |
|---|---|---|
| Group | Weight (g) | μg protein/30 min) |
| Affected | 23.8 ± 3.6 (n = 6) | 0.76 ± 0.04 (n = 6) |
| spf/ash | | |
| Treated | 33.8 ± 5.2 (n = 7) | 3.26 ± 0.41 (n = 7) |
| spf/ash | | |
| Unaffected | 36.8 ± 4.7 (n = 6) | 37.43 ± 2.63 (n = 6) |

In summary, these experiments demonstrate that the ASOR-coated nanocapsules can be injected ip and still exhibit liver-specific uptake in vivo in neonates. These experiments also demonstrate that single-stranded 45-mer oligonucleotides encapsulated in ASOR-coated nanocapsules can mediate site-directed repair of a genomic point mutation in hepatocytes. Also, these experiments demonstrate that neonatal gene repair 45-mer oligonucleotides targeted to the liver via the ASGPr receptors produce genomic change resulting in alterations in enzyme levels and improved phenotypes over animals receiving 5-times more 45-mer in nanocapsules that were not coated and, therefore, not targeted to ASGPr.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1-23. (canceled)

24. A composition of nanocapsules comprising at least one liver-specific targeting moiety and at least one cargo moiety, wherein said at least one targeting moiety is non-covalently associated with said nanocapsules, wherein said at least one cargo moiety is encapsulated by said nanocapsules, and wherein said nanocapsules have an average diameter of less than 50 nanometers.

25. The composition of claim 24, wherein said at least one targeting moiety is an asialoorosomucoid (ASOR) polypeptide or a hyaluronan (HA) polypeptide.

26. The composition of claim 24, wherein said at least one cargo moiety is a pharmaceutical agent.

27. The composition of claim 26, wherein said pharmaceutical agent is selected from the group consisting of a drug, a nucleic acid, a polypeptide, an anti-apoptotic agent, a chemoprotective agent, a chemopreventive agent, and an antiviral agent.

28. The composition of claim 27, wherein said nucleic acid is a plasmid expressing a therapeutic polypeptide.

29. The composition of claim 28, wherein said therapeutic polypeptide is selected from the group consisting of a Factor VII, a Factor VIII and a Factor IX polypeptide.

30. The composition of claim 27, wherein said nucleic acid is an oligonucleotide.

31. The composition of claim 27, wherein said polypeptide is selected from the group consisting of a Factor VII, a Factor VIII and a Factor IX polypeptide.

32. A method of targeting a pharmaceutical agent to liver cells, wherein the method comprises administering to a subject a composition of liver-targeted nanocapsules comprising:
a liver cell targeting moiety comprising an asialoorosomucoid (ASOR) polypeptide that-is non-covalently associated with the nanocapsules, and
a cargo moiety comprising a pharmaceutical agent that is encapsulated by the nanocapsules,
wherein the nanocapsules have an average diameter of less than 50 nanometers.

33. The method of claim 32, wherein said administering is intravenously or intraperitoneally.

34. The method of claim 32, wherein said liver cells are hepatocytes.

35. The method of claim 32, wherein said subject has a disease of the liver.

36. The method of claim 32, wherein the nanocapsules do not upregulate at least one of the groups consisting of Gadd45 and Gadd153 transcript levels.

37. The method of claim 32, wherein said pharmaceutical agent is selected from the group consisting of an anti-viral agent, a recombinogenic oligonucleotide, a siRNA oligonucleotide, an antisense molecule, an episomal DNA plasmid, a protein, and a drug.

38. The method of claim 35, wherein said disease is selected from the group consisting of Crigler-najjar syndrome, hemophilia A or B, alpha-1-antitrypsin deficiency, Wilson's disease, familial hypercholesterolemia, maple syrup urine disease, ornithine transcarbamylase deficiency, phenylketonuria, lysosomal storage diseases, glycogen storage diseases, peroxisome diseases, familial amyloidosis, cytochrome p450 diseases, bile acid synthesis defects, non-alcoholic fatty liver disease, non-alcoholic steatohepatitis; hepatitis A, B, C, D or E; cirrhosis, hemachomatosis, autoimmune hepatitis; cystic fibrosis, or hepatocellular carcinoma (HCC).

39. A method of mediating site-directed repair of a genomic mutation in liver cells of a subject, comprising:
administering the composition of claim 24 to said subject, wherein said nanocapsules are targeted to and bind to the liver cells, wherein said binding of said nanocapsules to said liver cells results in the delivery of said at least one cargo moiety to said liver cells, wherein said at least one cargo moiety is a single-stranded oligonucleotide, wherein delivery of said single-stranded oligonucleotide mediates site-directed repair of said genomic mutation in said liver cells of said subject.

40. The method of claim 39, wherein said liver cells are selected from the group consisting of hepatocytes and LSECs.

41. The method of claim 39, wherein said genomic mutation is a point mutation.

42. The method of claim 39, wherein said administering is intravenously or intraperitoneally.

43. The method of claim 39, wherein said liver cells, following said administration, exhibit altered levels or activity of a polypeptide relative to the levels or activity of said polypeptide in said liver cells prior to said administration, wherein said polypeptide is encoded by a nucleic acid sequence having homology to said single-stranded oligonucleotide.

44. The method of claim 39, wherein said subject, following said administration, exhibits improved phenotype compared to said subject prior to said administration.

45. The method of claim 43, wherein said polypeptide is a clotting factor.

46. The method of claim 45, wherein said clotting factor is Factor VII, Factor VIII, or Factor IX.
